(19) United States
(12) Patent Application Publication
(10) Pub. No.: US 2016/0097686 A1
(43) Pub. Date: Apr. 7, 2016

(54) REMOTELY CONTROLLED SEMI-AUTOMATIC MECHANIZED SAMPLING AND TEMPERATURE MEASURING PROBE APPARATUS FOR MOLTEN STEEL IN METALLURGICAL FURNACES

(71) Applicant: Edgar R. Wunsche, Whitby (CA)

(72) Inventor: Edgar R. Wunsche, Whitby (CA)

(21) Appl. No.: 14/876,582

(22) Filed: Oct. 6, 2015

Related U.S. Application Data
(63) Continuation of application No. 13/696,706, filed on Jan. 18, 2013, now abandoned.

(51) Int. Cl. G01K 13/02; G01K 1/14; F27D 21/00; G01N 33/20

(52) U.S. Cl. CPC G01K 13/02; G01N 33/206; G01K 1/14; F27D 21/0014

(57) ABSTRACT

A remotely controlled, semi-automatic sampling and temperature measuring apparatus for steelmaking comprises (i) an air-cooled main probe body carrying a retractable metallic pipe pole containing a plurality of measuring cardboard cartridges by means of a push-pull driving mechanism; (ii) a non-cooled half-moon circular probe housing comprising a rotating arm for inserting and removal of the main probe body from the furnace by means of a hydraulic rotating actuator; and (iii) a double sided frame arm connected to the furnace shell for rotating and repositioning the half-moon circular probe housing and the main probe body away from the furnace for replacing the cardboard cartridges.

### RELATED APPLICATIONS

The present application is a continuation of co-pending U.S. patent application Ser. No. 13/696,706 with a filing date of Jan. 18, 2013, which is a national stage entry of international application no. PCT/IB2010/001304, filed May 7, 2010, each of these applications being incorporated herein in their entirety by reference.

### BACKGROUND OF THE INVENTION

I. Field of the Invention

This invention relates to a multipurpose mechanized and semi-automatic remotely controlled measuring apparatus, able to be inserted into the high temperature operating chamber of the steelmaking metallurgical furnace through a small opening in the wall of the shell of the steelmaking metallurgical furnace located in an elevated location in the furnace shell wall, to be used for gaining accurate and reliable status information about molten steel processed in a steelmaking metallurgical furnace, in particular sample of the steel being processed, its temperature and carbon and oxygen parameters.

II. Description of the Prior Art

In addition to the still by steelmaking community used crude manually controlled and operated measuring device endangering the safety of the operating person, for gaining accurate and reliable status information about molten steel processed in a steelmaking metallurgical furnace, other known mechanized devices have identical serious and fundamental flaw: practically all of them are inserted into the steelmaking metallurgical furnace high temperature operating chamber to be submerged into the high temperature molten slag and steel through the wide open opening in shell wall known as “Slag Door”. Such operating procedure is fundamentally wrong for many purely technological operating reasons. The most negative flaw of this operating method is energy wasting and energy efficiency drastically reducing caused by unrestricted intake of cold ambient air; this air (about 80% N₂) is heated in the furnace high temperature operating chamber and then exhausted as high temperature “waste” gases. Further flaws of the “Open Slag Door” type of operation are: permanent, uncontrolled and premature discharge of not fully processed slag, high percentage of FeO in slag etc.

From the aspect of safety of the working environment in general, and specifically personal safety of the individual being assigned to use and operate the crude and in principle primitive poll type, manually controlled and operated measuring device for gaining accurate and reliable status information about molten steel is not by any means a tool having all the necessary safety features to be operated without endangering the operator. The poll type device introduced into the molten steel and slag through an open “Slag Door” and which therefore in spite of all possible precaution is in variety of ways extremely endangering the personal safety of the operating person. Some most common dangerous of situations, endangering the safety of the person operating the manually controlled measuring device are: Because of the actual size and dimensions of the poll the operator must be very close to the fully open large opening of the Slag Door, and by that being exposed to the extreme heat from the inner of the furnace. Furthermore, removing of the spent measuring cartridge from the steel pool of the manual type measuring device is frequently ending in severe burning of operator's hands.

With reference to these costly and unsafe steelmaking operating flaws it is therefore an object of the present invention to address disadvantages of the know methods for gaining accurate and reliable status information about molten steel processed in a steelmaking metallurgical furnace.

### SUMMARY OF THE INVENTION

In accordance with the first aspect, it is an object of the present invention to provide a remotely controlled, semi-automatic and fully mechanized sampling and temperature measuring apparatus comprising a main probe rugged linear and curved shape body of rectangular cross-section with intense liquid media cooling assuring problem free insertion and withdrawal from the high temperature operating chamber of the steelmaking metallurgical furnace through a high in the furnace shell vertical wall positioned relatively small opening.

Another object of the invention is to provide a highly functional, from the main probe body fast retracting and extending metallic pipe pole carrying different length card board cartridges with appropriate sensors at their free end to be submerged for a certain time period into the molten steel followed with rapid withdrawal as soon as the specific task of gaining accurate and reliable status information about molten steel processed in a steelmaking metallurgical furnace, in particular sample of the steel being processed, its temperature and carbon and oxygen contents has been accomplished.

Advantageously the apparatus also comprises hydraulically actuated and remotely controlled positive push-pull driving mechanism including permanent electric and compressed air connections allowing rapid and safe replacement of the metallic pipe pole.

A further object of the invention is to provide a non-cooled mechanical device subassembly rotating arm carrying the main probe body and assuring safe and reliable inserting and removing of the probe main body from the furnace interior in an advantageous rotating motion.

It is yet another object of this invention to provide a mechanical device subassembly for withdrawing the retracted rotating arm with main probe body from the furnace interior in generally rotating motion.

More advantageously, another mechanical device subassembly for rotating the rotating arm with main probe subassembly in generally vertical plane for adding or removing measuring card board cartridges from the metallic pole pipe; this mechanical device subassembly is permanently and advantageously mounted on the furnace shell vertical wall or the main platform, moving together with the metallurgical furnace.

The present invention overcomes and completely eliminates previously discussed use of the crude and in principle primitive, safety of work disregarding, manually controlled and operated measuring device for gaining accurate and reliable status information about molten steel processed in a steelmaking metallurgical furnace.

In particular, the feature of retracting of the steel pipe pole with already applied measuring cartridge is totally and completely preventing any kind of damage when inserted into metallurgical furnace interior as it is the case in many instances with other kind of probes.

### BRIEF DESCRIPTION OF THE DRAWING OF THE PREFERRED EMBODIMENTS OF THE INVENTION

For the purpose that the invention may be readily understood, applicable preferred embodiments of the invention will be described by way of example, with reference to the accompanying drawing wherein, with the numbers in the drawing are the same for the common components.

FIG. 1 illustrates an embodiment of a vertical section through the steel structure of the shell 1 of a metallurgical furnace lined with refractory 2 and containing molten steel 3; the vertical wall 4 of the shell 1 being provided with opening 5 serving for installation of the remotely controlled water cooled closure subassembly mechanism 6 permanently closed and it is operated only for the purpose of inserting of the subassembly of the linear and curved shape main probe body of rectangular and circular cross-section 10, into the high temperature operating chamber 16 of the steelmaking metallurgical furnace. The linear and curved shape main probe body of rectangular and circular cross-section 10 is an integral part of the major assembly of the multipurpose mechanized and semi-automatic remotely controlled measuring apparatus 15. With reference to the FIG. 1 the major assembly of the multipurpose mechanized and semi-automatic remotely controlled measuring apparatus 15 consists of five (5) functional sub-assemblies: the already mentioned rugged, linear and curved shape main probe body of rectangular and circular cross-section with intense liquid media cooling and with inside of the cooling circuit located compressed air piping 10; the non-cooled half-moon circular main probe body housing carrying remotely controlled rotating arm, actuated by a hydraulic rotating actuator for push-pull control driving mechanism 17; the remotely controlled push-pull driving mechanism including flexible permanent electric cable connection, all located inside of the main probe body 10 and controlled by the said hydraulic rotating actuator 18; the internally attached to the push-pull driving mechanism metallic pipe pole 22 for carrying cardboard cartridges 23; the double sided frame arm 19 connected to the metallurgical furnace shell vertical wall or furnace platform through a rotation bushing 20. Furthermore, while the arm 19 is rotating in vertical plane for less than 360° controlled by a linear motion hydraulic cylinder 21, at its free end the double sided frame arm 19 is carrying the half moon circular main probe body housing 17, said housing containing a second hydraulic rotator via shaft and bushing (not shown) allowing inter-depended rotation of the arm and the half moon circular main probe body housing for repositioning of the main probe subassembly away from the furnace high temperature chamber for replacement of the variety of consumable cardboard cartridges. The fast retracting and extending metallic pipe pole 22 carrying different length card board cartridges with appropriate sensors at their free end 23. The rugged, linear and curved shape only main sub-subassembly probe body of rectangular and circular cross-section 24 will be suited for intensive liquid media cooling and the inside of the cooling circuit area will be outfitted with compressed air piping 25. The end of the main sub-subassembly probe body of rectangular and circular cross-section 24, entering the high temperature operating chamber 16 of the steelmaking metallurgical furnace is provided with easy replaceable air cooled end protector 12. The other end of the main sub-subassembly probe body of rectangular and circular cross-section 24, is provided with boiling flange 25 connection for mechanical connecting to the counter plate flange 26 of the half moon circular main probe body housing 17. The double sided frame arm 19 will be connected to the metallurgical furnace shell vertical wall 4 or furnace platform through a rotation busing 27; furthermore, while the arm 19 is rotating in vertical plane for less than 360° controlled by a linear motion hydraulic cylinder 21, at its free end the double sided frame arm 19 is carrying the half moon circular main probe body housing 17, said housing containing a second hydraulic rotator via shaft and bushing 28 and 29 (for clarity not shown) allowing inter-depended rotation of the arm 19 and the half moon circular main probe body housing 17 for repositioning of the main probe subassembly away from the furnace high temperature chamber 16 for simple and most important safe and simple replacement of the consumable cardboards 23. Functionally, hydraulically activated rotary hydraulic actuator 18, via arm 28 propels the guided push-pull quasi roller chain driving mechanism 29; in turn, the guided push-pull quasi roller chain driving mechanism 29 causes reciprocating motion of the metallic pipe pole 11 for carrying cardboard cartridges 23 selected and appropriate velocity. The metallic pipe pole 11 carrying cardboard cartridges 23, is submerged for a certain time period into the molten steel 3 followed with rapid withdrawal as soon as the specific task of gaining accurate and reliable status information about molten steel processed in a steelmaking metallurgical furnace, in particular sample of the steel being processed, its temperature and carbon and oxygen contents has been accomplished. Furthermore, as shown in FIG. 1, while the arm 19 is rotating in vertical plane for less than 360° controlled by a linear motion hydraulic cylinder 21, at its free end the double sided frame arm 19 is carrying the half moon circular main probe body housing 17, said housing 17 containing a second hydraulic rotator 28 via shaft and bushing 29 (not shown) allowing inter-depended rotation of the arm and the half moon circular main probe body housing for repositioning of the main probe subassembly away from the furnace high temperature chamber for easy replacement of the variety of consumable cardboard cartridges 23. There are several functional Positions of the Remotely Controlled Semi-Automatic Mechanized Sampling and Temperature Measuring Probe Apparatus for Molten Steel in Metallurgical Furnaces. As illustrated in FIG. 1 position I. indicates the situation with linear and curved shape main probe body of rectangular and circular cross-section 10 inserted into the high temperature furnace chamber and metallic pipe pole 11 carrying cardboard cartridge 23 fully extended and submerged in the molten steel bath 3 during performing one the actual measuring functions. Position II. indicates the situation with metallic pipe pole 11 carrying cardboard cartridge 23 fully retracted into the inner of the linear and curved shape main probe body of rectangular and circular cross-section 10 the linear and curved shape main probe body of rectangular and circular cross-section 10 withdrawn from the high temperature chamber 16. Position III. indicates situation of of the Remotely Controlled Semi-Automatic Mechanized Sampling and Temperature Measuring Probe Apparatus for Molten Steel in Metallurgical Furnaces when the cartridges 23 are safely removed or applied to the attached to the metallic pipe pole 11. Position IV. indicates the parking position of the Apparatus. Position V. indicates cardboard the cartridge 23 applied to the metallic pipe pole 11 and submerged into the molten steel bath.

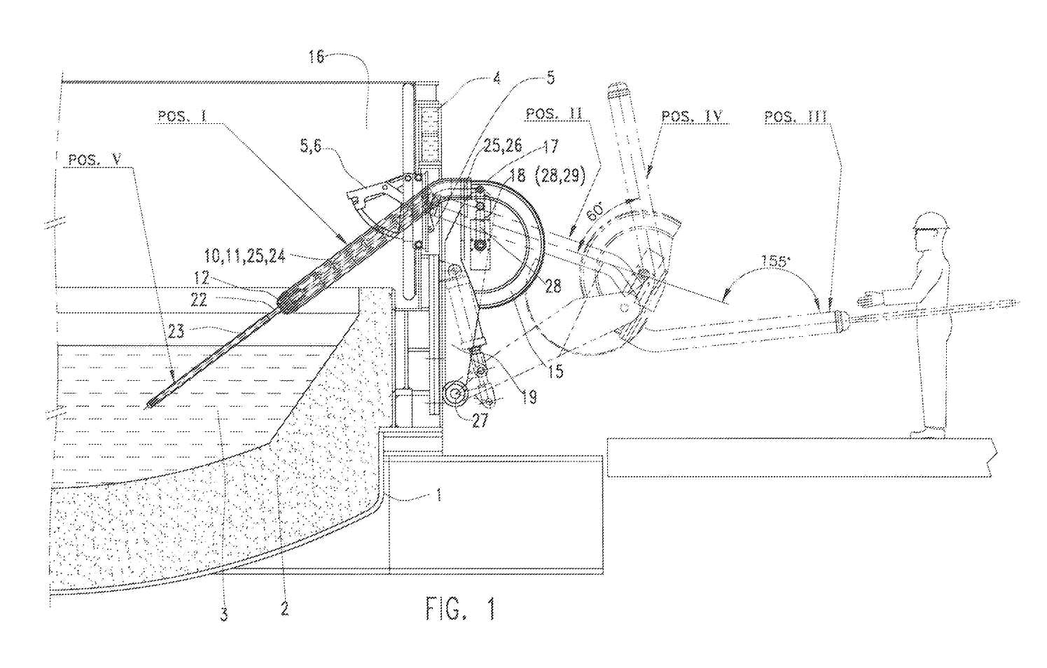

While the above description and accompanying various figure(s) have been made in connection with embodiments of the present invention as presently contemplated by the inventor, it is to be understood that modification and additions may be made to the described embodiment within the scope of the present invention. Therefore, the present invention should not be considered as being limited to the specific described embodiments, but construed in accordance with the appended claims.

1. A remotely controlled semi-automatic sampling and temperature measuring probe apparatus for molten steel in metallurgical furnaces, having several rotating and linear functional motions able to insert and withdraw from the high temperature operating chamber of the steelmaking metallurgical furnace through a high in the furnace shell vertical wall positioned relatively small opening having a highly functionalized from the body fast retracting and extending metallic pipe pole carrying different length of cardboard cartridges with appropriate sensors at their free end to be submerged for a certain time period into the molten steel followed with rapid withdrawal as soon as the specific task of gaining accurate and reliable status information about molten steel processed in a steelmaking metallurgical furnace, in particular sample of the steel being processed, its temperature and carbon and oxygen contents has been accomplished comprising:
a fundamental structural mechanical device subassembly permanently and advantageously mounted on the furnace vertical shell wall or main furnace platform, moving together with the metallurgical furnace for repositioning of the complete sub-ordinate mechanical device;
a sub-ordinate mechanical device assembly for rotating of the assembly of the rotating arm carrying the main probe subassembly in generally vertical plane for adding or removing measuring card board cartridges from the metallic pole pipe;
a sub-sub-assembly mechanical device with rotating arm carrying the main probe subassembly for rotating the main probe in generally vertical plane for adding or removing measuring card board cartridges from the metallic pole pipe;
a mechanical device subassembly rotating arm carrying the main probe body and assuring safe and reliable inserting and removing of the probe main body from the furnace interior in an advantageous rotating motion and in a combined vertical and eventually alterative horizontal direction;
a internally containing, non-cooled quasi roller chain driving mechanism, controlled through activation of the rotary hydraulic rotator for rapid inserting of the steep pole with replaceable cardboard cartridge at appropriate velocity with rapid removal from position of being submerged into molten steel bath;
a remotely controlled semi-automatic sampling and temperature measuring probe apparatus for molten steel in metallurgical furnaces and consisting of the partially water cooled main probe body having combined straight and curved portions, its straight portion cavity serving as a guide and protecting cover of mobile auxiliary components, while the cavity of the curved portion of the non water cooled main body of the apparatus contains circular quasi roller chain type push-pull drive mechanism converting rotary motion of the drive into linear motion.
